(19) United States
(12) Patent Application Publication
(10) Pub. No.: US 2019/0038421 A1
(43) Pub. Date: Feb. 7, 2019

(54) ARTIFICIAL ALPHA FEMORAL STEM PROSTHESIS

(71) Applicant: Beijing Lidakang Technology Co.,Ltd., Beijing (CN)

(72) Inventors: YAN WANG, BEIJING (CN); CHENGGONG ZHENG, BEIJING (CN); NING XU, BEIJING (CN); GUOQIANG ZHANG, BEIJING (CN)

(73) Assignee: Beijing Lidakang Technology Co.,Ltd.

(21) Appl. No.: 15/936,435

(22) Filed: Mar. 27, 2018

(30) Foreign Application Priority Data
Aug. 7, 2017 (CN) 201720976081.8
Dec. 6, 2017 (CN) 201721684843.3

(51) Int. Cl. A61F 2/36

(52) U.S. Cl. CPC A61F 2/367; A61F 2002/30828; A61F 2/3609

(57) ABSTRACT

The present disclosure provides an artificial alpha femoral stem prosthesis which includes a femoral stem cone, a femoral neck and a stem body connected integratedly, wherein a tool operating hole is arranged at upper end of the the femoral neck; the stem body comprises a proximal segment of the femoral stem, a middle segment of the femoral stem and a distal segment of the femoral stem, and there is a deflection angle between the femoral neck and the stem body.

### CROSS-REFERENCE TO RELATED APPLICATIONS

This application claims priority to Chinese Patent Application No. 201720976081.8 with a filing date of Aug. 7, 2017 and No. 201721684843.3 with a filing date of Dec. 6, 2017. The content of the aforementioned applications, including any intervening amendments thereto, are incorporated herein by reference.

### TECHNICAL FIELD

The present disclosure relates to the technical field of artificial prosthetic replacement, in particular to an artificial alpha (α) femoral stem prosthesis having a surface sprayed with microporous titanium coating.

### BACKGROUND OF THE PRESENT INVENTION

Congenital dysplasia of the hip (CDH) is a disease of abnormal hip joint. Some babies are born with this disease. The top of the thigh bone is not normally combined with the joint, which may lead to claudication and pain, and in more serious circumstances may even be disabled. If CDH is not treated in the early stage, the hip joint will be dysplastic, such as too large or too small anteversion angle of femora, or too shallow of an acetabulum, which will all cause difficulties in the normal movement of the hip after reaching adulthood.

The current way of treating CDH in adults and making them regain walking ability is to perform total hip arthroplasty by implanting the prosthesis into the hip to replace the original dysplastic hip joint.

The current method of correcting the femoral anteversion angle of the CDH patients mostly adopts modular femoral stem, which can assemble specific anteversion angle of the femoral stem for a specific patient using multiple components. Although specific anteversion angle of the femoral stem required by the specific patient can be assembled with multiple components using the manner of current modular femoral stem, after the modular products are implanted into the human body, components will suffer metal corrosion in the body due to the potential difference or other factors, which make the prosthesis must be removed. Modular femoral stems need to be configured according to the geometric shape of the femoral medullary cavity and anteversion angle of the femoral. The configuration during surgery will prolong the operative time, which may increase the sufferings of patients, and reduce the success rate of operation. Modular femoral stems need to be assembled when they are used, resulting in problems such as abrasion and osteolysis, which reduces the biocompatibility of prosthesis, and the manufacturing cost of huge stocks of components is suffered and the cost is high, which is not suitable for small manufacturers.

### SUMMARY OF PRESENT INVENTION

The technical problem to be solved by the disclosure is to provide a artificial alpha femoral stem prosthesis, to solve the problems of the current prosthesis such as abrasion between the components, osteolysis, poor biocompatibility of prosthesis, large inventory, high cost, configuration requirement of the prosthesis during surgery, long time operation, increase of the sufferings of patients, and low success rate of operation.

In order to solve the above technical problems, the embodiment of the disclosure provides an artificial alpha femoral prosthesis, which includes a femoral stem cone, a femoral neck and a stem body connected integratedly, wherein a tool operating hole is arranged at upper end of the the femoral neck. The stem body comprises a proximal segment of the femoral stem, a middle segment of the femoral stem and a distal segment of the femoral stem, and there is a deflection angle between the femoral neck and the stem body.

Preferably, the deflection angle between the femoral neck and the stem body is 0°˜35° or 145°˜180°

Preferably, the deflection angle between the femoral neck and the stem body is 0°, 15° or 35°, with a deflection direction towards left or towards right. The design of the angle is to solve the problem of metal corrosion of the modular femoral stem.

Preferably, the deflection angle between the central line of the stem body and the central line of the femoral neck is 120°˜140°.

Preferably, the deflection angle between the central line of the stem body and the central line of the femoral neck is 130°. The design of such angle is more in line with the structure of human physiological anatomy.

Preferably, the distal segment of the femoral stem has a structure of a highly polished bullet, avoiding collisions with the cortex, reducing the thigh pain and the cortical abrasion as much as possible.

Preferably, the height of the distal segment of the femoral stem is 14-22 mm, and the maximum cross-sectional diameter of the distal segment of the femoral stem is 7-13 mm. The maximum cross-sectional diameter of the distal segment of the femoral stem increases with an increment of 1 mm, and the height of the distal segment of the femoral stem increases accordingly. Taking the maximum cross-sectional diameter of the distal segment of the femoral stem having seven gradients as an example, corresponding deflection angle between the femoral neck and the stem body are 0°, 15°, and 35°, with the latter two towards left or right, and a total number of 35 products with different specifications can be generated for selection. Doctors can choose the product with an appropriate specification according to the needs of different patients.

Preferably, the cross section of the femoral neck is oval, which increases the range of activity of the prosthesis, and reduces the abrasion between the femoral neck and the total acetabular liner.

Preferably, the cross section of both the proximal segment of the femoral stem and the middle segment of the femoral stem is wedge-shaped, such that the stem body has a strong ability of anti-rotation, and also the blood circulation of the marrow cavity can be protected, and will not affect the blood supply of the marrow cavity.

Preferably, the outer surface of the proximal segment of the femoral stem is provided with a microgroove, wherein the bone graft can be automatically implanted during the prosthesis implantation, which ensures the stability of the prosthesis after it is implanted. The microgroove has a width of 1.0-1.1 mm, and a depth of 0.4-0.5 mm.

Preferably, the outer surface of the proximal segment of the femoral stem and the middle segment of the femoral stem are coated with microporous titanium coating, making the surface of the prosthesis become rough, which is more favorable for bone ingrowth, and enhancing the initial stability and the anti-rotation performance of the prosthesis.

Preferably, the thickness of the microporous titanium coating is 100-200 μm, and the porosity factor is 10-80%. The bonding strength of the microporous titanium coating is greater than or equal to 45 MPa, and the roughness of the microporous titanium coating is greater than or equal to 100 μm.

The beneficial effects of the technical scheme of the disclosure are as follows:

In the above scheme, the femoral neck in the disclosure has large range of activity, and the prosthesis implantation is stable and can effectively resist rotation, creating favorable conditions for ingrowth and proliferation of bone cells and soft tissue, improving the fixation effect of prosthesis in the human body, prolonging the service life of the prosthesis, improving the success rate of operation, reducing the postoperative pain of patients. The integrated prosthesis is adopted in the disclosure, without any need to assemble components, eliminating problems such as abrasion between components and osteolysis, reducing inventory, having a low cost, suitable for all kinds of large-scale or small-scale factory; the design of the femoral stem according to the disclosure conforms to the femoral stem for geometric shape of femoral medullary cavity of more than 90% patients; the anteversion angle of the disclosure is 0°, 15° or 35°, and patients can choose prosthesis of appropriate specifications during surgery according to their own anteversion angle, which may effectively shorten the operation time, reduce the pain of patients, and improve the success rate of the operation; the surface of the prosthesis in the present disclosure is sprayed with microporous titanium coating, which enhances biological compatibility of the prosthesis, and the strength and ductility and toughness match with human bone, thereby creating favorable conditions for ingrowth and proliferation of bone cells and soft tissue, so as to achieve a perfect match between the prosthesis and the patient's femora, especially suitable for developmental dysplasia of the hip (DDH) patients with congenital dislocation of the hip (CDH).

### DESCRIPTION OF THE DRAWINGS

FIG. 1 is a first schematic diagram of the structure of an artificial alpha femoral stem prosthesis according to the disclosure;

FIG. 2 is a second schematic diagram of the structure of the artificial alpha femoral stem prosthesis according to the disclosure;

FIG. 3 is a diagram of a deflection angle of the artificial alpha femoral stem prosthesis according to the disclosure.

### DESCRIPTION OF MAIN COMPONENT SIGN

1—femoral stem cone;

2—femoral neck;

3—stem body;

4—proximal segment of the femoral stem;

5—middle segment of the femoral stem;

6—distal segment of the femoral stem;

7—tool operating hole;

8—microgroove.

### DETAILED DESCRIPTION OF PREFERRED EMBODIMENTS

In order to make the technical problems to be solved, technical solutions and advantages more clearly, the following drawings and specific examples will be described in detail.

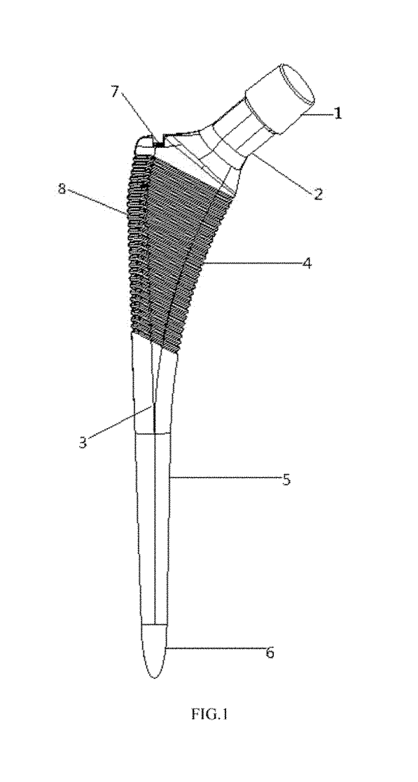

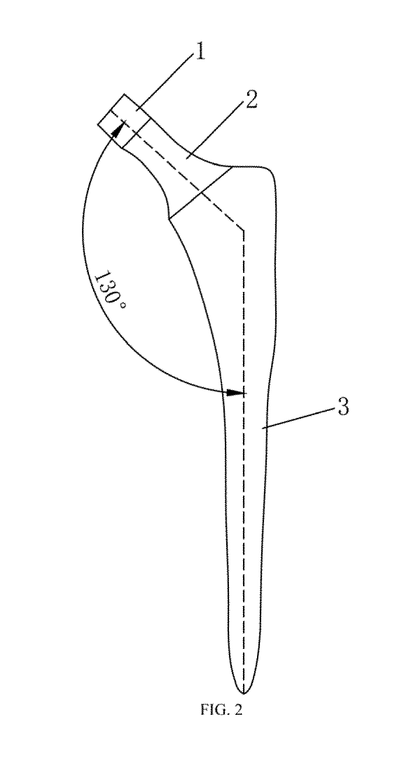

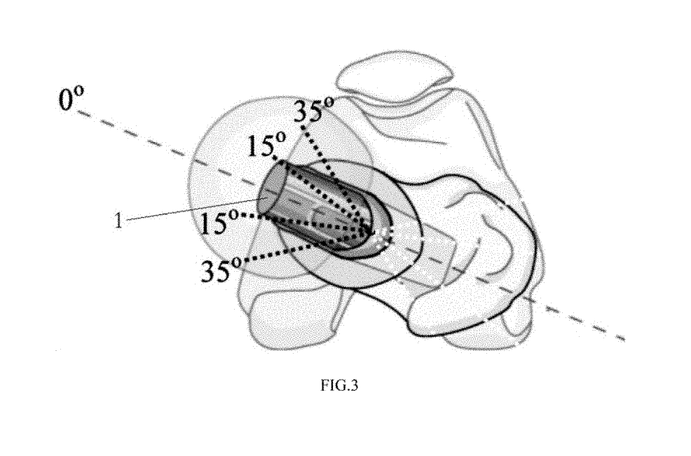

The preferred embodiment of the disclosure is shown in FIGS. 1 to 3.

An artificial alpha femoral prosthesis includes a femoral stem cone 1, a femoral neck 2 and a stem body 3 connected integratedly. The upper end of the femoral neck 2 is provided with a tool operating hole 7 into which a tool can insert to conduct an processing under an implantion operation. The stem body 3 comprises a proximal segment 4 of the femoral stem, a middle segment 5 of the femoral stem and a distal segment 6 of the femoral stem, and there is a deflection angle between the femoral neck 2 and the stem body 3, which is 0°˜35° or 145°˜180°.

Specifically, the deflection angle between the femoral neck and the stem body is an anteversion angle, which is 0°, 15° or 35°, with a direction towards left or towards right, and a total number of five angles. The design of the five angles is to solve the problems of metal corrosion of the modular femoral stem. The angle between the central line of the stem body 3 and the central line of the femoral neck 2 is 120°˜140°, with an optimal value of 130°. The design of such angle is more in line with the structure of human physiological anatomy. The cross section of the femoral neck 2 is oval, which increases the range of activities of the prosthesis, reduces the abrasion between the femoral neck 2 and total acetabular liner. The cross section of the proximal segment 4 of the femoral stem and the cross section of the middle segment 5 of the femoral stem is wedge-shaped, such that the stem body 3 has a strong ability of anti-rotation, and also the blood circulation of the marrow cavity can be protected, and will not affect the blood supply of the marrow cavity.

In this embodiment, the distal segment of the femoral stem 6 has a structure of a highly polished bullet, avoiding collision with the cortex, reducing the thigh pain and the cortical abrasion as much as possible. In addition, the height of the distal segment 6 of the femoral stem is 14-22 mm, and the maximum cross-sectional diameter of the distal segment 6 of the femoral stem is 7-13 mm. The maximum cross-sectional diameter of the distal segment 6 of the femoral stem increases with an increment of 1 mm, and the height of the distal segment of the femoral stem increases accordingly. Taking the maximum cross-sectional diameter of the distal segment of the femoral stem having seven gradients as an example, corresponding deflection angles between the femoral neck and the stem body are 0°,15° and 35° with the latter two towards left or right, and a total number of 35 products with different specifications can be generated for selection. Doctors can choose the product with an appropriate specification according to the needs of different patients.

Furthermore, the outer surface of the proximal segment 4 of the femoral stem is provided with a microgroove 8, wherein the bone graft can be automatically implanted during the prosthesis implantation, which ensures the stability of the prosthesis after it is implanted. The microgroove 8 has a width of 1.0-1.1 mm, and a depth of 0.4-0.5 mm. The outer surface of the proximal segment 4 of the femoral stem and the middle segment 5 of the femoral stem are coated with microporous titanium coating, making the surface of the prosthesis become rough, which is more favorable for bone ingrowth, and enhancing the initial stability and the anti-rotation performance of the prosthesis. The thickness of the microporous titanium coating is 100-200 μm. and the porosity factor is 10-80%. The bonding strength of the microporous titanium coating is greater than or equal to 45 MPa, and the roughness of the microporous titanium coating is greater than or equal to 100 μm.

The femoral neck 2 in the disclosure has large range of activity, and the prosthesis implantation is stable and effective antirotation, creating favorable conditions for ingrowth and proliferation of bone cells and soft tissue, improving the fixation effect of prosthesis in the human body, prolonging the service life of the prosthesis, improving the success rate of operation, reducing the postoperative pain of patients.

The integrated prosthesis is adopted in the disclosure, without any need to assemble components, eliminating problems such as abrasion between components and osteolysis, reducing inventory, having a low cost, suitable for all kinds of large-scale or small-scale factory; the design of the femoral stem according to the disclosure conforms to the femoral stem for geometric shape of femoral medullary cavity of more than 90% patients; the anteversion angle in the disclosure is 0°, 15° or 35°, and patients can choose prosthesis of appropriate specifications during surgery according to their own anteversion angle, which may effectively shorten the operation time, reduce the pain of patients, and improve the success rate of the operation. The outer surface of the prosthesis in the present disclosure is sprayed with microporous titanium coating, which enhances biological compatibility of the prosthesis, and the strength and ductility and toughness match with human bone, thereby creating favorable conditions for ingrowth and proliferation of bone cells and soft tissue, so as to achieve a perfect match between the prosthesis and the patient's femora, especially suitable for developmental dysplasia of the hip (DDH) patients with congenital dislocation of the hip (CDH).

The above is the preferred embodiment of the disclosure, it should be pointed out that for those of ordinary skill in the art, some improvement and polishing can be made without departing from the principle of the disclosure, and these improvements should also be regarded as the scope of protection of the disclosure.

We claim:

1. An artificial alpha femoral stem prosthesis, comprising a femoral stem cone, a femoral neck and a stem body connected integratedly, where a tool operating hole is arranged at upper end surface of the femoral neck, characterized in that the stem body comprises a proximal segment of the femoral stem, a middle segment of the femoral stem and a distal segment of the femoral stem, and there is a deflection angle between the femoral neck and the stem body.

2. The artificial alpha femoral stem prosthesis according to claim 1, characterized in that, the deflection angle between the femoral neck and the stem body is 0°˜35°, or 145°˜180°.

3. The artificial alpha femoral stem prosthesis according to claim 1, characterized in that, the deflection angle between the femoral neck and the stem body is 0°, 15° or 35°, and a deflection direction is toward left or toward right.

4. The artificial alpha femoral stem prosthesis according to claim 1, characterized in that, an angle between a central line of the stem body and a central line of the femoral neck is 120°˜140°.

5. The artificial alpha femoral stem prosthesis according to claim 1, characterized in that, an angle between a central line of the stem body and a central line of the femoral neck is 130°.

6. The artificial alpha femoral stem prosthesis according to claim 1, characterized in that, the distal segment of the femoral stem has a structure of high polished bullet.

7. The artificial alpha femoral stem prosthesis according to claim 4, characterized in that, a height of the distal segment of the femoral stem is 14-22 mm, and a diameter of a maximum cross section of the distal segment of the femoral stem is 7-13 mm.

8. The artificial alpha femoral stem prosthesis according to claim 5, characterized in that, a cross section of the femoral neck is oval.

9. The artificial alpha femoral stem prosthesis according to claim 6, characterized in that, the cross section of the proximal segment of the femoral stem and the middle segment of the femoral stem is wedge-shaped.

10. The artificial alpha femoral stem prosthesis according to claim 7, characterized in that, a outer surface of the proximal segment of the femoral stem is provided with a microgroove having a width of 1.0-1.1 mm, and a depth of 0.4-0.5 mm.
